(19) United States
(12) Patent Application Publication
(10) Pub. No.: US 2004/0040098 A1
(43) Pub. Date: Mar. 4, 2004

(54) Compositions for the oxidation dyeing of keratin fibers and dyeing processes using these compositions

(75) Inventors: Gerard Lang, Saint Prix (FR); Alain Lagrange, Coupvray (FR)

Correspondence Address: FINNEGAN, HENDERSON, FARABOW, GARRETT & DUNNER, LLP, 1300 I STREET, NW, WASHINGTON, DC, 20005, US

(73) Assignee: L'OREAL

(21) Appl. No.: 10/651,229

(22) Filed: Aug. 29, 2003

Related U.S. Application Data
(63) Continuation of application No. 09/583,724, filed on May 30, 2000, now abandoned.

(30) Foreign Application Priority Data
May 28, 1999 (FR) 9906799

(51) Int. Cl. A61K 7/13

(52) U.S. Cl. 8/405

(57) ABSTRACT

The subject of the invention is ready-to-use compositions for the oxidation dyeing of keratin fibers, and in particular human keratin fibers such as the hair, comprising, in a medium which is suitable for dyeing, at least one oxidation base chosen from para-phenylenediamines, double bases, para-aminophenols and heterocyclic bases, at least one enzyme of 2-electron oxidoreductase type in the presence of at least one donor for the enzyme, and at least one enzymatic mediator, and the oxidation dyeing processes using these compositions.

The invention relates to ready-to-use compositions for the oxidation dyeing of keratin fibers, and in particular human keratin fibers such as the hair, comprising, in a medium which is suitable for dyeing, at least one oxidation base chosen from para-phenylenediamines, double bases, para-aminophenols and heterocyclic bases, at least one enzyme of 2-electron oxidoreductase type in the presence of at least one donor for the enzyme, and at least one enzymatic mediator, and to the dyeing processes using these compositions.

It is known to dye keratin fibers, and in particular human hair, with dye compositions containing oxidation dye precursors, in particular ortho- or para-phenylenediamines, ortho- or para-aminophenols, and heterocyclic bases, which are generally known as oxidation bases. Oxidation dye precursors, or oxidation bases, are colorless or weakly colored compounds which, when combined with oxidizing products, can give rise to colored compounds and dyes by the process of oxidative condensation.

It is known that the shades obtained with these oxidation bases can be varied by combining them with couplers or coloration modifiers, these modifiers being chosen in particular from aromatic meta-diamines, meta-aminophenols, meta-diphenols and certain heterocyclic compounds. The variety of molecules used as oxidation bases and couplers allows a wide variety of colors to be obtained.

It is desirable that the so-called “permanent” coloration obtained by means of these oxidation dyes satisfy a certain number of requirements. For example, it will preferably have no toxicological drawbacks, allow shades to be obtained in the desired intensity, and show good resistance with respect to external agents (light, bad weather, washing, permanent waving, perspiration and rubbing).

It is also desirable for the dyes to allow grey hair to be covered, and to be as unselective as possible, i.e., they should allow only the smallest possible differences in coloration along the same keratin fiber, which may in fact be differently sensitized (i.e., damaged) between its tip and its root.

The oxidation dyeing of keratin fibers is generally carried out in alkaline medium, in the presence of hydrogen peroxide. However, the use of alkaline media in the presence of hydrogen peroxide has the drawbacks of causing an appreciable degradation of the fibers, as well as considerable bleaching of keratin fibers, which is not always desirable.

The oxidation dyeing of keratin fibers can also be carried out using oxidizing systems other than hydrogen peroxide, such as enzymatic systems. Thus, it has already been proposed to dye keratin fibers, in particular in patent application EP-A-0 310 675, the disclosure of which is incorporated by reference herein, with compositions comprising an oxidation base and optionally a coupler, in combination with enzymes such as pyranose oxidase, glucose oxidase or uricase, in the presence of a donor for the enzymes. However, although these dyeing processes are carried out under conditions that do not cause a degradation of the keratin fibers that is comparable with that generated by the dyeing operations carried out in the presence of hydrogen peroxide, they lead to colorations which are not entirely satisfactory, in particular with regard to their intensity and their resistance with respect to the various attacking factors to which the hair may be subjected.

It has also already been proposed, in particular in patent application WO 98/40471, the disclosure of which is incorporated by reference herein, to dye keratin fibers with compositions comprising an oxidation dye precursor, an enzyme of laccase type (4-electron oxido-reductase) and an enzymatic mediator. However, the colorations obtained using these compositions are not always satisfactory with regard to the intensity and the resistance of the colorations obtained with respect to the various attacking factors to which the hair may be subjected.

The use of an enzymatic mediator is also well known in the paper industry for bleaching and delignifying paper pulp, for example in H. P. Call et al., Journal of Biotechnology 53 (1997) 163-202, the disclosure of which is incorporated by reference herein.

The inventors have now discovered that it is possible to obtain novel dyes, that are capable of leading to strong colorations without giving rise to any significant degradation of the keratin fibers, that are unselective and that show good resistance to the various attacking factors to which the fibers may be subjected, by combining at least one oxidation base chosen from para-phenylenediamines, para-aminophenols, double bases and heterocyclic bases, at least one enzyme of 2-electron oxidoreductase type in the presence of at least one donor for the enzyme, and at least one enzymatic mediator.

This discovery forms the basis of the present invention.

A subject of the invention is thus a ready-to-use composition for the oxidation dyeing of keratin fibers, and in particular human keratin fibers such as the hair, characterized in that it comprises, in a medium which is suitable for dyeing:

at least one oxidation base chosen from:

i) para-phenylenediamines of formula (I) below, and the acid addition salts thereof:

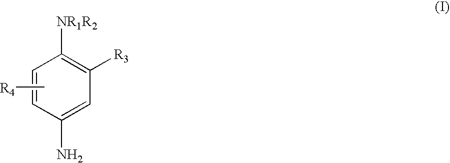

in which:

R₁ and R₂, which may be identical or different, represent a hydrogen atom or a C₁-C₄ alkyl or C₁-C₄ monohydroxyalkyl radical;

R₃ represents a hydrogen or halogen atom or a C₁-C₄ alkyl or C₁-C₄ monohydroxyalkyl radical;

R₄ represents a hydrogen atom or a C₁-C₄ alkyl radical;

ii) double bases;

iii) para-aminophenols of formula (II) below, and the acid addition salts thereof:

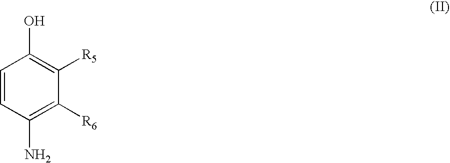

in which:

R₅ and R₆, which may be identical or different, represent a hydrogen or halogen atom or a C₁-C₄ alkyl, C₁-C₄ monohydroxyalkyl, (C₁-C₄)alkoxy-(C₁-C₄)alkyl; C₁-C₄ aminoalkyl or monohydroxy(C₁-C₄)alkylamino(C₁-C₄)alkyl radical,

it being understood that at least one of the radicals R₅ and R₆ represents a hydrogen atom; and

iv) heterocyclic bases;

at least one enzyme of 2-electron oxidoreductase type;

at least one donor for the enzyme; and

at least one enzymatic mediator capable of increasing the enzymatic activity of the 2-electron oxidoreductase.

The ready-to-use dye composition in accordance with the invention rapidly gives strong colorations which have low selectivity and excellent properties of resistance, both with respect to atmospheric agents such as light and bad weather, and with respect to perspiration and the various treatments to which the hair may be subjected.

A subject of the invention is also a process for the oxidation dyeing of keratin fibers using this ready-to-use dye composition.

The 2-electron oxidoreductase(s) used in the ready-to-use dye composition in accordance with the invention can be chosen in particular from pyranose oxidases, glucose oxidases, glycerol oxidases, lactate oxidases, pyruvate oxidases, uricases, choline oxidases, sarcosine oxidases and bilirubin oxidases.

According to the invention, the 2-electron oxidoreductase is preferably chosen from uricases of animal, microbiological and biotechnological origin. By way of example, mention may be made in particular of the uricase extracted from boar's liver, the uricase from Arthrobacter globiformis and the uricase from Aspergillus flavus.

The 2-electron oxidoreductase(s) can be used in pure crystalline form or in a form diluted in a diluent which is inert with respect to the said 2-electron oxidoreductase.

The 2-electron oxidoreductase(s) in accordance with the invention preferably represent(s) from 0.01 to 20% by weight approximately relative to the total weight of the ready-to-use dye composition, and even more preferably from 0.1 to 5% by weight approximately.

According to the invention, the term “donor” means the various substrates participating in the functioning of the 2-electron oxidoreductase(s).

The nature of the donor (or substrate) for the enzyme varies as a function of the nature of the 2-electron oxidoreductase used. For example, donors for the pyranose oxidases which may be mentioned are D-glucose, L-sorbose and D-xylose; a donor for the glucose oxidases which may be mentioned is D-glucose; donors for the glycerol oxidases which may be mentioned are glycerol and dihydroxyacetone; donors for the lactate oxidases which may be mentioned are lactic acid and its salts; donors for the pyruvate oxidases which may be mentioned are pyruvic acid and its salts; donors for the uricases which may be mentioned are uric acid and its salts; donors for the choline oxidases which may be mentioned are choline and its acid addition salts, such as choline hydrochloride and betain aldehyde; donors for the sarcosine oxidases which may be mentioned are sarcosine, N-methyl-L-leucine, N-methyl-D,L-alanine and N-methyl-D,L-valine; and finally, a donor for the bilirubin oxidases which may be mentioned is bilirubin.

The donor(s) (or substrate(s)) used in accordance with the invention preferably represent(s) from 0.01 to 20% by weight approximately relative to the total weight of the ready-to-use dye composition and even more preferably from 0.1 to 5% approximately.

The enzymatic mediator(s) used in the ready-to-use composition in accordance with the invention make(s) it possible to increase the enzymatic activity of the oxidoreductase used, and can be chosen, for example, from the compounds of formula (Ill) below, and the possible tautomeric forms thereof:

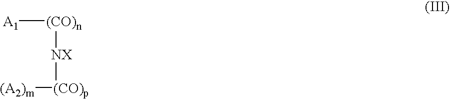

in which:

A₁ and A₂, which may be identical or different, represent:

a) a saturated or unsaturated, linear or branched aliphatic radical containing from 1 to 30 carbon atoms, it being possible for the aliphatic radical to be substituted with one or more hydroxyl, halo, sulpho, carboxyl, nitro or phenyl radicals;

b) a heterocyclic radical containing from 1 to 4 hetero atoms and from 5 to 10 ring members, it being possible for the heterocyclic radical to be substituted with one or more C₁-C₄ alkyl, halo, phenyl, hydroxyl or C₇-C₁₀ aralkyl radicals;

c) an aromatic radical comprising from 6 to 10 ring members, it being possible for the aromatic radical to be substituted with one or more C₁-C₄ alkyl, halo, sulpho, carboxyl; nitro, hydroxyl or nitroso radicals;

it also being possible for the nitrogen atom of the group NX to form, with the groups A₁-(CO)ₙ and A₂-(CO)ₚ, a heterocycle comprising from 5 to 18 ring members, it being possible for the heterocycle to be substituted with one or more C₁-C₄ alkyl, hydroxyl, phenyl, halo, sulpho, carboxyl or nitro radicals;

X represents a group —OH, ═O, ═S, →O or →S; and

m, n and p, independently of each other, are integers equal to 0 or 1.

Among the enzymatic mediators of formula (III) above, mention may be made in particular of hydroxylamine, N,N-dipropylhydroxylamine, N,N-diisopropylhydroxylamine, phenylhydroxylamine, N-acetylhydroxylamine, 1-phenyl-1H-1,2,3-triazole 1-oxide, 2,4,5-triphenyl-2H-1,2,3-triazole 1-oxide, 1-hydroxybenzotriazole, 1-hydroxy-benzotriazolesulphonic acid, 1-hydroxybenzimidazole, N-hydroxyphthalimide, N-hydroxy-succinimide, quinoline N-oxide, isoquinoline N-oxide, 1-hydroxypiperidine, violuric acid, 4-hydroxy-3-nitrosocoumarin, 1,3-dimethyl-5-nitrosobarbituric acid, 1-nitroso-2-naphthol, 2-nitroso-l-naphthol-4-sulphonic acid, 2-nitroso-l -naphthol, 1-nitroso-2-naphthol-3,6-di-sulphonic acid and 2,4-dinitroso-1,3-dihydroxybenzene.

The enzymatic mediator(s) used in the ready-to-use composition in accordance with the invention can also be chosen from syringic acid and its esters; acetosyringone; syringaldehyde; para-hydroxycinnamic acid; vanillin; 7-hydroxycoumarin; 2,4-dichlorophenol; para-hydroxybenzenesulphonate; 2,2′-azinobis(3-ethylbenzothiazoline-6-sulphonate); phenothiazines such as 10-methylphenothiazine; benzidines such as 3,3′-di-methylbenzidine; amino derivatives of 2-naphthalenesulphonic acid; L-tyrosine; ferulic acid; caffeic acid; chlorogenic acid and sinapic acid.

The enzymatic mediator(s) used in the ready-to-use dye composition in accordance with the invention preferably represent(s) from 0.0001 to 5% by weight approximately relative to the total weight of the ready-to-use dye composition and even more preferably from 0.005 to 0.5% by weight approximately.

Among the para-phenylenediamines of formula (I) described above which can be used as oxidation bases in the ready-to-use dye composition in accordance with the invention, mention may be made in particular of para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)-amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, and the acid addition salts thereof.

Among the double bases which can be used as oxidation bases in the ready-to-use dye composition in accordance with the invention, mention may be made in particular of compounds comprising at least two aromatic nuclei which bear several amino and/or hydroxyl groups.

Among the said double bases which may be mentioned more particularly are the compounds of formula (IV) below, and the acid addition salts thereof:

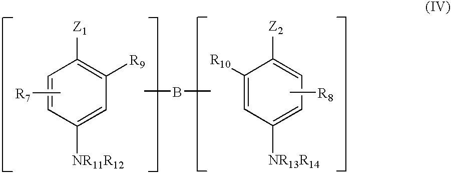

in which:

Z₁ and Z₂, which may be identical or different, represent a hydroxyl or —NH₂ radical which may be substituted with a C₁-C₄ alkyl radical or with a linker arm B;

the linker arm B represents a linear or branched alkylene chain containing from 1 to 14 carbon atoms, which can be interrupted and/or can end with one or more nitrogenous groups and/or with one or more hetero atoms such as oxygen, sulphur or nitrogen atoms, and which may be optionally substituted with one or more hydroxyl or C₁-C₆ alkoxy radicals;

R₇ and R₈ represent a hydrogen or halogen atom, a C₁-C₄ alkyl, C₁-C₄ monohydroxyalkyl, C₂-C₄ polyhydroxyalkyl or C₁-C₄ aminoalkyl radical or a linker arm B; and

R₉, R₁₀, R₁₁, R₁₂, R₁₃ and R₁₄, which may be identical or different, represent a hydrogen atom, a linker arm B or a C₁-C₄ alkyl radical;

it being understood that the compounds of formula (IV) comprise only one linker arm B per molecule.

Among the nitrogenous groups in formula (IV) which may be mentioned in particular are amino, mono(C₁-C₄)alkylamino, di(C₁-C₄)alkylamino, tri(C₁-C₄)alkylamino, monohydroxy(C₁-C₄)alkylamino, imidazolinium and ammonium radicals.

Among the double bases of formula (IV) above which may be mentioned more particularly are N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-amino-phenyl)tetramethylenediamine, N,N′-bis(4-methylaminophenyl)tetramethylenediamine, N,N′-bis(ethyl)-N,N′-bis(4′-amino-3′-methylphenyl)ethylenediamine, 1,8-bis(2,5-diaminophenoxy)-3,5-dioxaoctane, and the acid addition salts thereof.

Among the double bases of formula (IV), N,N′-bis(β-hydroxyethyl)-N,N′-bis(4′-aminophenyl)-1,3-diaminopropanol, 1,8-bis(2,5-diaminophenoxy)-3,5-dioxaoctane, or one of the acid addition salts thereof, are particularly preferred.

Among the para-aminophenols of formula (II) described above which can be used as oxidation bases in the ready-to-use dye composition in accordance with the invention, mention may be made more particularly of para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, 4-amino-2-fluorophenol, and the acid addition salts thereof.

Among the heterocyclic bases which can be used as oxidation bases in the ready-to-use dye composition in accordance with the invention, mention may be made more particularly of pyridine derivatives, pyrimidine derivatives, pyrazole derivatives, and the acid addition salts thereof.

Among the pyridine derivatives which may be mentioned more particularly are the compounds described, for example, in patents GB 1 026 978 and GB 1 153 196, the disclosures of which are incorporated by reference herein, such as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine, 3,4-diaminopyridine, and the acid addition salts thereof.

Among the pyrimidine derivatives which may be mentioned more particularly are the compounds described, for example, in German patent DE 2 359 399, Japanese patents JP 88-169 571 and JP 91-10659, and patent application WO 96/15765, the disclosures of which are incorporated by reference herein, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine and 2,5,6-triaminopyrimidine, and pyrazolopyrimidine derivatives such as those mentioned in patent application FR-A-2 750 048, the disclosure of which is incorporated by reference herein, and among which mention may be made of pyrazolo[1,5-a]pyrimidine-3,7-diamine, 2,5-dimethylpyrazolo[1,5-a]pyrimidine-3,7-diamine, pyrazolo[1,5-a]pyrimidine-3,5-diamine, 2,7-dimethylpyrazolo[1,5-a]pyrimidine-3,5-diamine, 3-amino-pyrazolo[1,5-a]pyrimidin-7-ol, 3-aminopyrazolo[1,5-a]pyrimidin-5-ol, 2-(3-aminopyrazolo-[1,5a]pyrimidin-7-ylamino)ethanol, 2-(7-aminopyrazolo[1,5-a]pyrimidin-3-ylamino)ethanol, 2-[(3-aminopyrazolo[1,5-a]pyrimidin-7-yl )(2-hydroxyethyl )amino]ethanol, 2-[(7-aminopyrazolo[1,5-a]pyrimidin-3-yl)(2-hydroxyethyl)amino]ethanol, 5,6-dimethyl-pyrazolo[1,5-a]pyrimidine-3,7-diamine, 2,6-dimethylpyrazolo[1,5-a]pyrimidine-3,7-diamine, 2,5,N7, N7-tetramethylpyrazolo[1-,5-a]pyrimidine-3,-7-diamine, the addition salts thereof, the tautomeric forms thereof when a tautomeric equilibrium exists, and the acid addition salts thereof.

Among the pyrazole derivatives which may be mentioned more particularly are the compounds described in patents DE 3 843 892 and DE 4 133 957 and patent applications WO 94/08969, WO 94/08970, FR-A-2 733 749 and DE 195 43 988, the disclosures of which are incorporated by reference herein, such as 4,5-diamino-1-methylpyrazole, 3,4-diaminopyrazole, 4,5-diamino-1-(4′-chlorobenzyl)pyrazole, 4,5-diamino-1,3-dimethyl-pyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-tri-aminopyrazole, 3,5-diamino-1-methyl-4-methylaminopyrazole, 3,5-diamino4-(β-hydroxyethyl)amino-1-methylpyrazole, and the acid addition salts thereof.

According to the invention, the oxidation base(s) preferably represent(s) from 0.0005 to 12% by weight approximately relative to the total weight of the ready-to-use dye composition, and even more preferably from 0.005 to 6% by weight approximately.

According to one preferred embodiment, the ready-to-use dye composition in accordance with the invention may further contain one or more couplers and/or one or more direct dyes, particularly to modify the shades or to enrich them with glints.

Among the couplers which may be additionally present in the ready-to-use dye composition in accordance with the invention, mention may be made in particular of meta-aminophenols, meta-phenylenediamines, meta-diphenols, heterocyclic couplers such as, for example, indole derivatives, indolene derivatives, sesamol and derivatives thereof, pyridine derivatives, pyrazolotriazole derivatives and pyrazolones, and the acid addition salts thereof.

These couplers are chosen more particularly from 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino4-(β-hydroxyethylamino)-1-methoxybenzene, 1,3-diaminobenzene, 1,3-bis(2,4-diaminophenoxy)propane, sesamol, 1-amino-2-methoxy-4,5-methylenedioxybenzene, α-naphthol, 6-hydroxyindole, 4-hydroxyindole, 4-hydroxy-N-methylindole, 6-hydroxyindolene, 2,6-dihydroxy4-methylpyridine, 1H-3-methylpyrazol-5-one, 1-phenyl-3-methylpyrazol-5-one, and the acid addition salts thereof.

When they are present, these additional couplers preferably represent from 0.0001 to 10% by weight approximately relative to the total weight of the ready-to-use dye composition, and even more preferably from 0.005 to 5% by weight approximately.

The acid addition salts which can be used in the context of the dye compositions of the invention (oxidation bases and couplers) are chosen in particular from hydrochlorides, hydrobromides, sulphates, tartrates, lactates and acetates.

The medium which is suitable for dyeing for the ready-to-use dye composition in accordance with the invention generally comprises water or a mixture of water and at least one organic solvent to dissolve the compounds which would not be sufficiently water-soluble. Organic solvents which may be mentioned, for example, are C₁-C₄ alkanols, such as ethanol and isopropanol; glycerol; glycols and glycol ethers such as 2-butoxyethanol, propylene glycol, propylene glycol monomethyl ether, diethylene glycol monoethyl ether and monomethyl ether; aromatic alcohols such as benzyl alcohol or phenoxyethanol; and similar products and mixtures thereof.

The solvents may be present in proportions preferably ranging from 1 to 40% by weight approximately relative to the total weight of the ready-to-use dye composition, and even more preferably from 5 to 30% by weight approximately.

The pH of the ready-to-use composition in accordance with the invention is chosen such that the enzymatic activity of the 2-electron oxidoreductase is sufficient. It generally ranges from 3 to 11 approximately, and preferably from 4 to 9 approximately. It can be adjusted to the desired value using acidifying or basifying agents usually used in the dyeing of keratin fibers.

Among the acidifying agents which may be mentioned, for example, are inorganic or organic acids such as hydrochloric acid, orthophoshoric acid, sulphuric acid, carboxylic acids such as acetic acid, tartaric acid, citric acid and lactic acid, and sulphonic acids.

Among the basifying agents which may be mentioned, for example, are aqueous ammonia, alkaline carbonates, alkanolamines such as mono-, di- and triethanolamines, 2-methyl-2-amino-1-propanol and derivatives thereof, sodium hydroxide, potassium hydroxide and the compounds of formula (V) below:

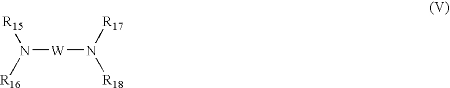

in which W is a propylene residue optionally substituted with a hydroxyl group or a C₁-C₄ alkyl radical, and R₁₅, R₁₆, R₁₇ and R₁₈, which may be identical or different, represent a hydrogen atom or a C₁-C₄ alkyl or C₁-C₄ hydroxyalkyl radical.

The ready-to-use dye composition in accordance with the invention can also contain various adjuvants conventionally used in compositions for dyeing the hair, such as anionic, cationic, nonionic, amphoteric or zwitterionic surfactants or mixtures thereof; anionic, cationic, nonionic, amphoteric or zwitterionic polymers or mixtures thereof; inorganic or organic thickeners; antioxidants; enzymes other than the 2-electron oxidoreductases used in accordance with the invention, such as, for example, peroxidases; penetrating agents; sequestering agents; fragrances; buffers; dispersants; conditioners such as, for example, volatile or non-volatile, modified or unmodified silicones; film-forming agents; ceramides; preserving agents; and opacifiers.

Needless to say, a person skilled in the art will take care to select this or these optional additional compound(s) such that the advantageous properties intrinsically associated with the ready-to-use dye composition in accordance with the invention are not, or are not substantially, adversely affected by the addition(s) envisaged.

The ready-to-use dye composition in accordance with the invention can be in various forms, such as in the form of liquids, creams or gels, which are optionally pressurized, or in any other form which is suitable for dyeing keratin fibers, and in particular human hair. In the present invention, the oxidation dyes and the 2-electron oxido-reductase(s) are present in the same ready-to-use composition and, consequently, it is desirable that the composition be free of gaseous oxygen, so as to avoid any premature oxidation of the oxidation dye(s).

According to the invention, the term “alkyl” can mean a linear alkyl when there are one or two carbon atoms, or a linear, branched or cyclic alkyl when there are three or more carbon atoms.

A subject of the invention is also a process for dyeing keratin fibers, and in particular human keratin fibers such as the hair, using the ready-to-use dye composition as defined above.

According to this process, at least one ready-to-use dye composition as defined above is applied to the fibers, at an application temperature ranging from room temperature to 80° C., for a period which is sufficient to develop the desired coloration. Preferably, the fibers are then rinsed, optionally washed with shampoo and then dried.

The application temperature preferably ranges from room temperature to 60° C., and even more preferably from 35° C. to 50° C.

The time which is sufficient to develop the coloration on the keratin fibers generally ranges from 1 to 60 minutes, and even more specifically from 5 to 30 minutes.

According to one specific embodiment of the invention, the process comprises: first, separately storing a composition (A) comprising, in a medium which is suitable for dyeing, at least one oxidation base as defined above, and a composition (B) comprising, in a medium which is suitable for dyeing, at least one enzyme of 2-electron oxidoreductase type in the presence of at least one donor for the enzyme, and at least one enzymatic mediator capable of increasing the enzymatic activity of the 2-electron oxidoreductase; then mixing them together at the time of use; and finally applying the mixture to the keratin fibers.

Another subject of the invention is a multi-compartment dyeing device or dyeing “kit” or any other multi-compartment packaging system, comprising a first compartment which contains composition (A) as defined above, and a second compartment which contains composition (B) as defined above. These devices may be equipped with a means for applying the desired mixture to the hair, such as the devices described in patent FR-2 586 913, the disclosure of which is incorporated by reference herein.

The example which follows is intended to illustrate the invention without, however, limiting its scope.

### EXAMPLE OF DYEING

The ready-to-use dye composition below was prepared (contents in grams):

| COMPOSITION | 1 |
|---|---|
| para-Phenylenediamine (oxidation base of formula (I) ) | 0.324 |
| 1,3-Dihydroxybenzene (coupler) | 0.33 |
| 1-Hydroxybenzotriazole (enzymatic mediator) | 0.1 |
| Uricase from Arthrobacter globiformis at 20 International Units | 1.5 |
| (I.U.)/mg, sold by the company Sigma | |
| Uric acid | 1.5 |
| Dye support (*) | (*) |
| Demineralized water q.s. | 100 g |
| (*): Dye support: | |
| Hydroxyethylcellulose sold under the trade name | 1.0 g |
| NATROSOL 250 HHR ® by the company Aqualon | |
| 96° ethanol | 20 g |
| 2-Methyl-2-amino-1-propanol q.s. | pH 9.5 |

(*): Dye support:

The ready-to-use dye composition described above was applied to locks of natural grey hair containing 90% white hairs, for 30 minutes at a temperature of 30° C. The hair was then rinsed, washed with a standard shampoo and then dried.

The hair was dyed in a matte light chestnut shade.

What is claimed is:

1. A composition for the oxidation dyeing of keratin fibers, comprising, in a medium suitable for dyeing:
at least one oxidation base chosen from:
i) para-phenylenediamines of formula (I) and the acid addition salts thereof: 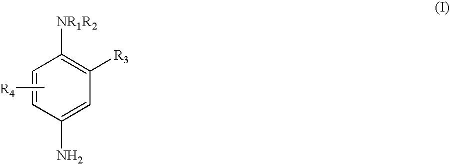
wherein:
R₁ and R₂, which may be identical or different, are chosen from a hydrogen atom, a C₁-C₄ alkyl radical, and a C₁-C₄ monohydroxyalkyl radical;
R₃ is a hydrogen or halogen atom or a C₁-C₄ alkyl or C₁-C₄ monohydroxyalkyl radical;
R₄ is a hydrogen atom or a C₁-C₄ alkyl radical;
ii) double bases;
iii) para-aminophenols of formula (II) and the acid addition salts thereof: 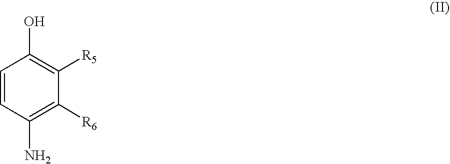
wherein:
R₅ and R₆, which may be identical or different, are chosen from a hydrogen atom, a halogen atom, a C₁-C₄ alkyl radical, a C₁-C₄ monohydroxyalkyl radical, a (C₁-C₄)alkoxy(C₁-C₄)alkyl radical, a C₁-C₄ aminoalkyl radical and a monohydroxy (C₁-C₄)alkylamino(C₁-C₄)alkyl radical, with the proviso that at least one of R₅ and R₆ is a hydrogen atom; and
iv) heterocyclic bases;
at least one enzyme of 2-electron oxidoreductase type;
at least one donor for said at least one enzyme of 2-electron oxidoreductase type; and
at least one enzymatic mediator capable of increasing the enzymatic activity of said at least one enzyme of 2-electron oxidoreductase type.

2. A composition according to claim 1, wherein said keratin fibers are human keratin fibers.

3. A composition according to claim 2, wherein said human keratin fibers are hair.

4. A composition according to claim 1, wherein said at least one enzyme of 2-electron oxidoreductase type is chosen from pyranose oxidases, glucose oxidases, glycerol oxidases, lactate oxidases, pyruvate oxidases, uricases, choline oxidases, sarcosine oxidases and bilirubin oxidases.

5. A composition according to claim 4, wherein said at least one enzyme of 2-electron oxidoreductase type is chosen from uricases of animal, microbiological and biotechnological origin.

6. A composition according to claim 1, wherein said at least one enzyme of 2-electron oxidoreductase type is present in an amount ranging from 0.01 to 20% by weight relative to the total weight of the composition.

7. A composition according to claim 6, wherein said at least one enzyme of 2-electron oxidoreductase type is present in an amount ranging from from 0.1 to 5% by weight relative to the total weight of the composition.

8. A composition according to claim 1, wherein said at least one donor is present in an amount ranging from 0.01 to 20% by weight relative to the total weight of the composition.

9. A composition according to claim 8, wherein said at least one donor is present in an amount ranging from 0.1 to 5% by weight relative to the total weight of the composition.

10. A composition according to claim 1, wherein said at least one enzymatic mediator is chosen from the compounds of formula (Ill) below, and the tautomeric forms thereof: 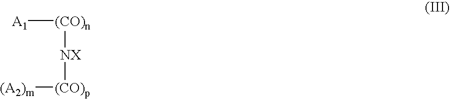
wherein:
A₁ and A₂, which may be identical or different, are chosen from:
a) a saturated or unsaturated, linear or branched aliphatic radical containing from 1 to 30 carbon atoms, wherein said aliphatic radical is optionally substituted with one or more hydroxyl, halo, sulpho, carboxyl, nitro or phenyl radicals;
b) a heterocyclic radical containing from 1 to 4 hetero atoms and from 5 to 10 ring members, wherein said heterocyclic radical is optionally substituted with one or more C₁-C₄ alkyl, halo, phenyl, hydroxyl or C₇-C₁₀ aralkyl radicals; and
c) an aromatic radical comprising from 6 to 10 ring members, wherein said aromatic radical is optionally substituted with one or more C₁-C₄ alkyl, halo, sulpho, carboxyl, nitro, hydroxyl or nitroso radicals;
or wherein the groups A₁-(CO)ₙ and A₂-(CO)ₚ may form, together with the nitrogen atom of the group NX, a heterocycle comprising from 5 to 18 ring members, wherein said heterocycle is optionally substituted with one or more C₁-C₄ alkyl, hydroxyl, phenyl, halo, sulpho, carboxyl or nitro radicals;
X represents a group —OH, ═O, ═S, →O or →S; and
m, n and p, which may be identical or different, are integers equal to 0 or 1.

11. A composition according to claim 10, wherein said compounds of formula (III) are chosen from hydroxylamine, N,N-dipropylhydroxylamine, N,N-diisopropylhydroxyl-amine, phenylhydroxylamine, N-acetylhydroxylamine, 1-phenyl-1H-1,2,3-triazole 1-oxide, 2,4, 5-triphenyl-2H-1,2,3-triazole 1-oxide, 1-hydroxybenzotriazole, 1-hydroxy-benzotriazolesulphonic acid, 1-hydroxybenzimidazole, N-hydroxyphthalimide, N-hydroxy-succinimide, quinoline N-oxide, isoquinoline N-oxide, 1-hydroxypiperidine, violuric acid, 4-hydroxy-3-nitrosocoumarin, 1,3-dimethyl-5-nitrosobarbituric acid, 1-nitroso-2-naphthol, 2-nitroso-1-naphthol-4-sulphonic acid, 2-nitroso-1-naphthol, 1-nitroso-2-naphthol-3,6-di-sulphonic acid and 2,4-dinitroso-1,3-dihydroxy-benzene.

12. A composition according to claim 1, wherein said at least one enzymatic mediator is chosen from syringic acid and its esters, acetosyringone, syringaldehyde, para-hydroxycinnamic acid, vanillin, 7-hydroxycoumarin, 2,4-dichlorophenol, para-hydroxybenzenesulphonate, 2,2′-azinobis(3-ethylbenzothiazoline-6-sulphonate), phenothiazines, benzidines, amino derivatives of 2-naphthalenesulphonic acid, L-tyrosine, ferulic acid, caffeic acid, chlorogenic acid and sinapic acid.

13. A composition according to claim 12, wherein said at least one enzymatic mediator is 10-methylphenothiazine.

14. A composition according to claim 12, wherein said at least one enzymatic mediator is 3,3′-dimethylbenzidine.

15. A composition according to claim 1, wherein said at least one enzymatic mediator is present in an amount ranging from 0.0001 to 5% by weight approximately relative to the total weight of the composition.

16. A composition according to claim 15, wherein said at least one enzymatic mediator is present in an amount ranging from 0.005 to 0.5% by weight approximately relative to the total weight of the composition.

17. A composition according to claim 1, wherein said para-phenylenediamines of formula (I) are chosen from para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)-amino-2-methylaniline, 4-N,N-bis(β-hydroxyethyl)amino-2-chloroaniline, 2-β-hydroxyethyl-para-phenylenediamine, 2-fluoro-para-penylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, and the acid addition salts thereof.

18. A composition according to claim 1, wherein said double bases are chosen from the compounds of formula (IV) below, and the acid addition salts thereof: 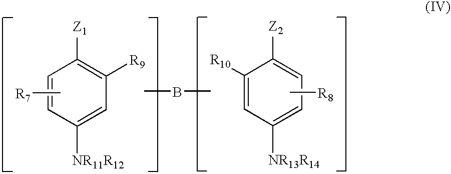
wherein:
Z₁ and Z₂, which may be identical or different, are chosen from a hydroxyl radical and a —NH₂ radical optionally substituted with a C₁-C₄ alkyl radical or with a linker arm B;
the linker arm B is a linear or branched alkylene chain comprising from 1 to 14 carbon atoms, which can be interrupted and/or can end with one or more nitrogenous groups and/or with one or more hetero atoms, and which may be optionally substituted with one or more hydroxyl or C₁-C₆ alkoxy radicals;
R₇ and R₈ are chosen from a hydrogen atom, a halogen atom, a C₁-C₄ alkyl radical, a C₁-C₄ monohydroxyalkyl radical, a C₂-C₄ polyhydroxyalkyl radical, a C₁-C₄ aminoalkyl radical and a linker arm B; and
R₉, R₁₀, R₁₁ , R₁₂, R₁₃ and R₁₄, which may be identical or different, are chosen from a hydrogen atom, a linker arm B and a C₁-C₄ alkyl radical;
with the overall proviso that said compounds of formula (IV) comprise only one linker arm B per molecule.

19. A composition according to claim 18, wherein said hetero atoms are chosen from oxygen, sulphur and nitrogen atoms.

20. A composition according to claim 18, wherein said compounds of formula (IV) are chosen from N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-amino-phenyl)tetramethylenediamine, N,N′-bis(4-methylaminophenyl)tetramethylenediamine, N,N′-bis(ethyl)-N,N′-bis(4′-amino-3′-methylphenyl)ethylenediamine, 1,8-bis(2,5-diamino-phenoxy)-3,5-dioxaoctane, and the acid addition salts thereof.

21. A composition according to claim 1, wherein said para-aminophenols of formula (II) are chosen from para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, 4-amino-2-fluorophenol, and the acid addition salts thereof.

22. A composition according to claim 1, wherein said heterocyclic bases are chosen from pyridine derivatives, pyrimidine derivatives, pyrazole derivatives, and the acid addition salts thereof.

23. A composition according to claim 1, wherein said at least one oxidation base is present in an amount ranging from 0.0005 to 12% by weight relative to the total weight of the composition.

24. A composition according to claim 23, wherein said at least one oxidation base is present in an amount ranging from 0.005 to 6% by weight relative to the total weight of the composition.

25. A composition according to claim 1, wherein said composition is a ready-to-use composition for the oxidation dyeing of keratin fibers.

26. A composition according to claim 1, wherein said composition further comprises at least one coupler and/or at least one direct dye.

27. A composition according to claim 26, wherein said at least one coupler is chosen from meta-aminophenols, meta-phenylene-diamines, meta-diphenols, heterocyclic couplers, and the acid addition salts thereof.

28. A composition according to claim 27, wherein said at least one coupler is chosen from 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, sesamol, 1-amino-2-methoxy-4,5-methylenedioxybenzene, α-naphthol, 6-hydroxyindole, 4-hydroxyindole, 4-hydroxy-N-methylindole, 6-hydroxyindolene, 2,6-dihydroxy-4-methylpyridine, 1H-3-methylpyrazol-5-one, 1-phenyl-3-methylpyrazol-5-one, and the acid addition salts thereof.

29. A composition according to claim 1, wherein said acid addition salts are chosen from hydrochlorides, hydrobromides, sulphates, tartrates, lactates, and acetates.

30. A composition according to claim 1, wherein said composition has a pH ranging from 4 to 9.

31. A process for the oxidation dyeing of keratin fibers, comprising applying to said fibers, at an application temperature ranging from room temperature and 80° C., and for a time period sufficient to develop the desired coloration, at least one dyeing composition comprising, in a medium suitable for dyeing:
at least one oxidation base chosen from:
i) para-phenylenediamines of formula (I) and the acid addition salts thereof: 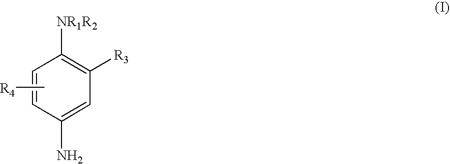
wherein:
R₁ and R₂, which may be identical or different, are chosen from a hydrogen atom, a C₁-C₄ alkyl radical, and a C₁-C₄ monohydroxyalkyl radical;
R₃ is a hydrogen or halogen atom or a C₁-C₄ alkyl or C₁-C₄ monohydroxyalkyl radical;
R₄ is a hydrogen atom or a C₁-C₄ alkyl radical;
ii) double bases;
iii) para-aminophenols of formula (II) and the acid addition salts thereof: 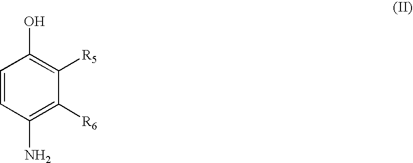
wherein:
R₅ and R₆, which may be identical or different, are chosen from a hydrogen atom, a halogen atom, a C₁-C₄ alkyl radical, a C₁-C₄ monohydroxyalkyl radical, a (C₁-C₄)alkoxy(C₁-C₄)alkyl radical, a C₁-C₄ aminoalkyl radical and a monohydroxy (C₁-C₄)alkylamino(C₁-C₄)alkyl radical, with the proviso that at least one of R₁ and R₆ is a hydrogen atom; and
iv) heterocyclic bases;
at least one enzyme of 2-electron oxidoreductase type;
at least one donor for said at least one enzyme of 2-electron oxidoreductase type; and
at least one enzymatic mediator capable of increasing the enzymatic activity of said at least one enzyme of 2-electron oxidoreductase type.

32. A process according to claim 31, wherein said keratin fibers are human keratin fibers.

33. A process according to claim 32, wherein said human keratin fibers are hair.

34. A process according to claim 31, wherein said application temperature ranges from 35° C. to 50° C.

35. A process according to claim 31, wherein said time period sufficient to develop the desired coloration ranges from 1 to 60 minutes.

36. A process according to claim 35, wherein said time period ranges from 5 to 30 minutes.

37. A process according to claim 31, wherein said process further comprises the steps of:
separately storing a first composition comprising, in a medium appropriate for dyeing, said at least one oxidation base, and a second composition comprising, in a medium appropriate for dyeing, said at least one enzyme of 2-electron oxidoreductase type, said at least one donor, and said at least one enzymatic mediator;
mixing said first composition and said second composition at the time of use; and
applying the mixture to said keratin fibers.

38. A multi-compartment dyeing device, comprising:
at least one first compartment which contains at least one composition comprising, in a medium appropriate for dyeing, at least one oxidation base chosen from:
i) para-phenylenediamines of formula (I) and the acid addition salts thereof: 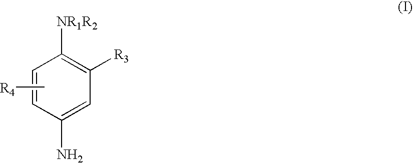
wherein:
R₁ and R₂, which may be identical or different, are chosen from a hydrogen atom, a C₁-C₄ alkyl radical, and a C₁-C₄ monohydroxyalkyl radical:
R₃ is a hydrogen or halogen atom or a C₁-C₄ alkyl or C₁-C₄ monohydroxyalkyl radical;
R₄ is a hydrogen atom or a C₁-C₄ alkyl radical;
ii) double bases;
iii) para-aminophenols of formula (II) and the acid addition salts thereof: 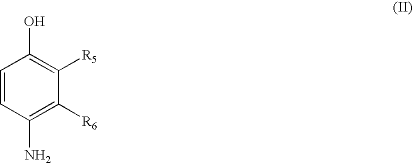
wherein:
R₅ and R₆, which may be identical or different, are chosen from a hydrogen atom, a halogen atom, a C₁-C₄ alkyl radical, a C₁-C₄ monohydroxyalkyl radical, a (C₁-C₄)alkoxy(C₁-C₄)alkyl radical, a C₁-C₄ aminoalkyl radical and a monohydroxy (C₁-C₄)alkylamino(C₁-C₄)alkyl radical, with the proviso that at least one of R₅ and R₆ is a hydrogen atom; and
iv) heterocyclic bases; and
at least one second compartment which contains at least one composition comprising, in a medium appropriate for dyeing:
at least one enzyme of 2-electron oxidoreductase type;
at least one donor for said at least one enzyme of 2-electron oxidoreductase type; and
at least one enzymatic mediator capable of increasing the enzymatic activity of said at least one enzyme of 2-electron oxidoreductase type.
